(19) United States
(12) Patent Application Publication
(10) Pub. No.: US 2021/0361151 A1
(43) Pub. Date: Nov. 25, 2021

(54) ENDOSCOPIC CAMERA SHEATH

(71) Applicant: ARTHREX, INC., Naples, FL (US)

(72) Inventors: Ryan Kellar, Naples, FL (US); Brittany Foslien, Naples, FL (US); Richard Taft, Naples, FL (US); Sanjeev Kakar, Rochester, MN (US)

(21) Appl. No.: 17/325,656

(22) Filed: May 20, 2021

Related U.S. Application Data
(60) Provisional application No. 63/027,580, filed on May 20, 2020.

(51) Int. Cl. A61B 1/05; A61B 1/005; A61B 1/018; A61B 1/06

(52) U.S. Cl. CPC A61B 1/05; A61B 1/0051; A61B 2217/005; A61B 1/0623; A61B 1/018

(57) ABSTRACT

An endoscopic camera system having a camera with a shaft coupled to a handpiece; and a sheath with hollow tube coupled to a body. The tube has: a proximal portion coupled to the body; a bend at a distal end of the proximal portion; and a distal portion distal to the bend. The sheath is configured to deflect the shaft and the sheath is rotatable around the shaft to change a direction of the deflection.

### CROSS REFERENCE TO RELATED APPLICATION

This application claims priority of U.S. Provisional Patent Application No. 63/027,580, filed on May 20, 2020, entitled ENDOSCOPIC CAMERA SHEATH, the entire contents of which is hereby incorporated herein by reference.

### BACKGROUND

The present disclosure relates to devices used in endoscopic surgery and, more particularly, to sheaths for endoscopic cameras for manipulating the field of view of an endoscopic camera system.

### SUMMARY

The present disclosure relates to an endoscopic camera system. Endoscopic camera systems provide a surgeon with a view of an area of the body being worked on without the necessity of fully opening up the area, thereby allowing for less invasive surgical procedures. Miniaturization of image sensors allows image sensors to be placed near the distal tip of an endoscopic camera so that the image sensor rotates along with the endoscopic camera to change the field of view. Rotation of the image sensor can be disorienting to a surgeon. There is a need for an improved endoscopic camera system that is less disorienting to a surgeon.

In an implementation, an endoscopic camera system may have a camera with a shaft coupled to a handpiece; and a sheath having a hollow tube coupled to a body. The tube may have: a proximal portion coupled to the body; a bend at a distal end of the proximal portion; and a distal portion distal to the bend. The sheath may be configured to deflect the shaft. The sheath may be rotatable about the shaft to change a direction of the deflection.

In an implementation, the shaft may comprise a proximal end and a distal end and an image sensor may be positioned proximal to the distal end. The shaft may have optics to provide light to the image sensor and the optics may provide a field of view approximately along a longitudinal axis of the shaft. The shaft may have a diameter of less than about 2.0 mm and the hollow tube further may have a lumen with a diameter of less than about 2.1 mm.

The distal portion may have a longitudinal axis at an angle of between about 5 degrees and about 90 degrees relative to a longitudinal axis of the proximal portion. The distal portion may have a longitudinal axis of about 70 degrees, 45 degrees or 30 degrees relative to a longitudinal axis of the proximal portion. Additionally, the distal portion may have a longitudinal axis at an angle of between about 7.5 degrees and about 15 degrees relative to a longitudinal axis of the proximal portion. Additionally, the distal portion may have a longitudinal axis at an angle of between about 9 degrees and about 11 degrees relative to a longitudinal axis of the proximal portion. The bend may have a radius of from about 12 mm to about 38 mm. Additionally, the bend may have a radius of from about 25 mm to about 32 mm.

The bend may have a radius of from about 12 mm to about 38 mm. Additionally, the bend may have a radius of from about 25 mm to about 32 mm. The endoscopic camera system may also have a flexible obturator configured to be removably inserted into the sheath.

In some implementations, a sheath for an endoscopic camera system may have a body and a hollow tube, the hollow tube having a proximal portion coupled to the body; a bend at a distal end of the proximal portion; and a distal portion distal to the bend. The hollow tube may comprise a lumen with a diameter of less than about 2.1 mm.

The distal portion may have a longitudinal axis at an angle of between about 5 degrees and about 90 degrees relative to a longitudinal axis of the proximal portion. The distal portion may have a longitudinal axis of about 70 degrees, 45 degrees or 30 degrees relative to a longitudinal axis of the proximal portion. Additionally, the distal portion may have a longitudinal axis at an angle of between about 7.5 degrees and about 15 degrees relative to a longitudinal axis of the proximal portion. Additionally, the distal portion may have a longitudinal axis at an angle of between about 9 degrees and about 11 degrees relative to a longitudinal axis of the proximal portion. The bend may have a radius of from about 12 mm to about 38 mm. Additionally, the bend may have a radius of from about 25 mm to about 32 mm.

In an implementation, a method for imaging a surgical field with a camera having a shaft coupled to a handpiece and a sheath having a hollow tube coupled to a body, the tube having a proximal portion coupled to the body; a bend at a distal end of the proximal portion; and a distal portion distal to the bend, comprises the steps of: positioning the sheath in a surgical field; inserting the camera shaft into the sheath to deflect the shaft; and moving the sheath relative to the shaft to change an angle of the deflection to change the field of view. Additionally, the method may have the steps of inserting an obturator into the sheath prior to positioning the sheath in a surgical field; and removing the obturator from the sheath prior to inserting the camera shaft into the sheath. Additionally, the method may have the step of connecting at least one of a fluid source and a suction source to the sheath.

These and other features are described below.

### BRIEF DESCRIPTION OF THE DRAWINGS

The features, aspects and advantages of the present invention will become better understood with regard to the following description, appended claims and accompanying figures wherein:

FIG. 1 is a schematic diagram of a camera system according to an implementation;

FIG. 2 is a perspective view of a sheath according to an implementation; and

FIG. 3 is a side elevation view of the sheath of FIG. 2;

FIG. 4 is a side elevation view of the sheath of FIG. 2;

FIG. 5 is a side elevation view of the sheath of FIG. 2;

FIG. 6 is a side elevation view of a camera and a schematic cross sectional elevation view of a sheath body, according to implementations;

FIG. 7 is a side elevation view of a camera and a schematic cross sectional elevation view of a sheath showing the camera shaft inside the sheath according to an implementation;

FIG. 8 is a side elevation view of an obturator usable with the sheath of FIG. 2; and

FIG. 9 is a flow chart illustrating a method of using an endoscopic camera system according to an implementation.

### DETAILED DESCRIPTION

In the following description of the preferred implementations, reference is made to the accompanying drawings which show by way of illustration specific implementations in which the invention may be practiced. Wherever possible, the same reference numbers will be used throughout the drawings to refer to the same or like parts. It is to be understood that other implementations may be utilized and structural and functional changes may be made without departing from the scope of this disclosure.

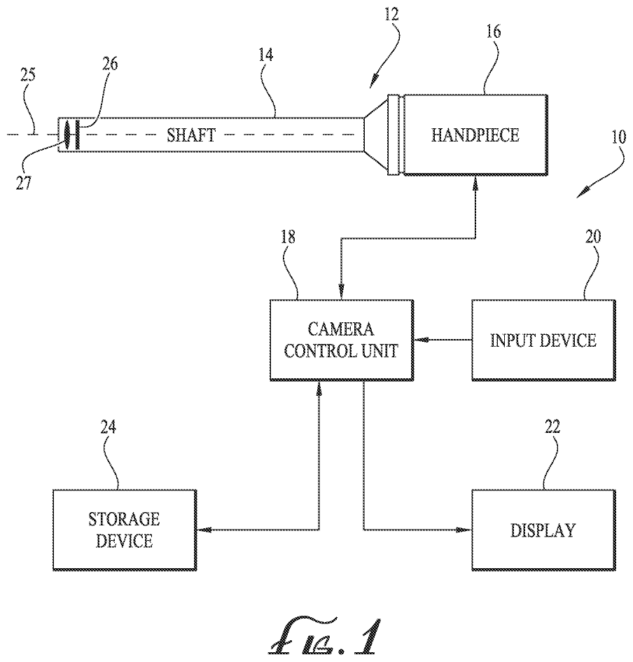

With reference to FIG. 1, an endoscopic camera system 10 according to an implementation has a camera 12. The camera 12 may have a shaft 14 couplable to a handpiece 16. The handpiece 16 may be connectable to a camera control unit 18 (“CCU” or “camera controller”). The handpiece 16 and the camera control unit 18 may be connected via wire or wirelessly. The camera control unit 18 may be connectable to at least one input device 20 such as a mouse, keyboard, touchpad, or touchscreen monitor. Additionally, the camera control unit may be connectable to a display 22 and a storage device 24 for storing images.

An image sensor 26 may be positioned inside the shaft 14 and proximal to a distal tip of the shaft. The shaft 14 may also include optics 27 for directing light to the image sensor 26. The position of the image sensor 26 and optics 27 may provide a field of view approximately along a longitudinal axis of the shaft 14 (approximately 0 degrees relative to the longitudinal axis) so that the image field is directly in front of the distal tip of the shaft. In some implementations, the optics may provide an image at a non-zero angle relative to the longitudinal axis of the shaft 14, such as for example, about 30 degrees or about 70 degrees.

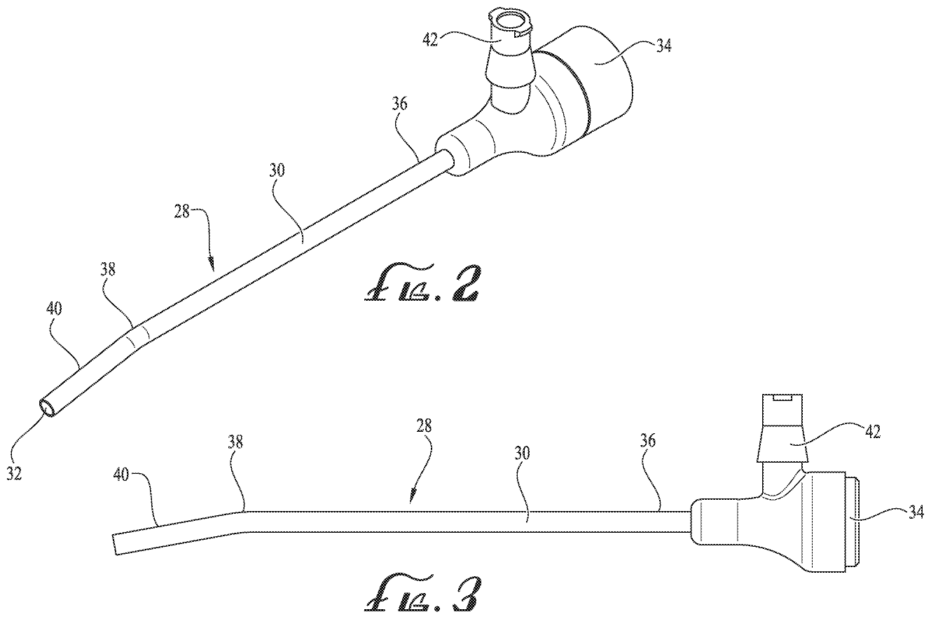

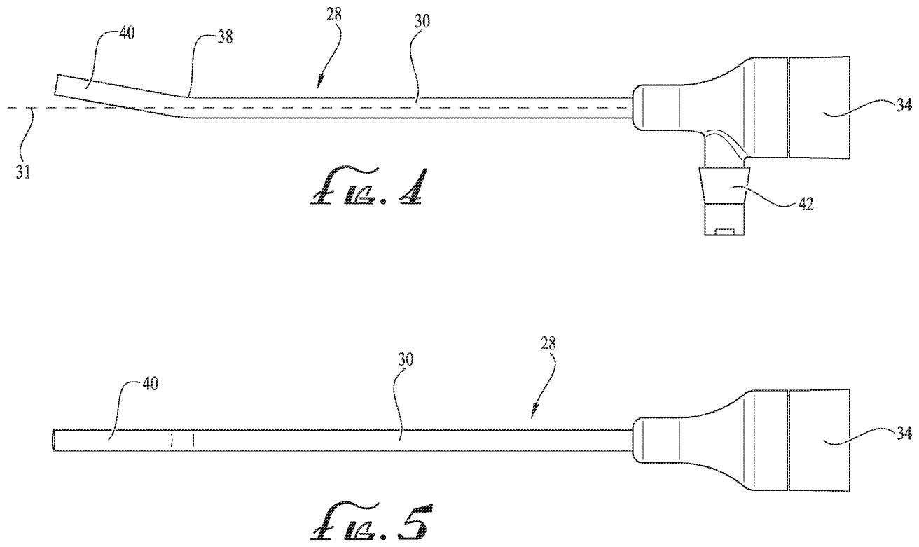

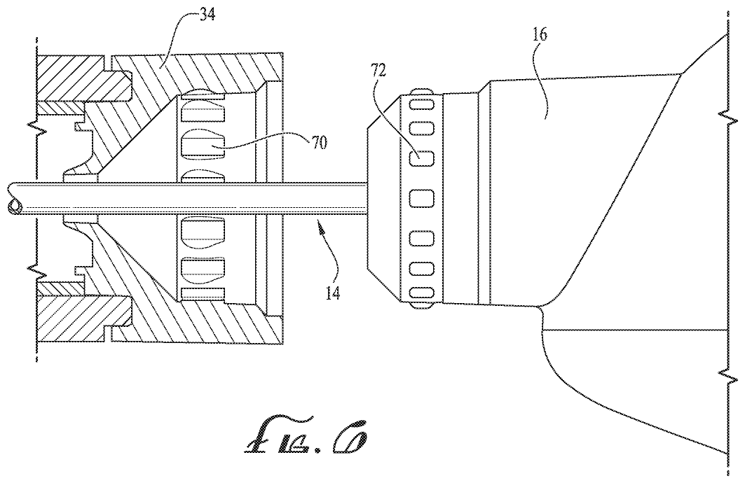

With reference to FIGS. 2 to 5, a pre-bent sheath 28 according to an implementation will now be further described. Although the term “sheath” is used throughout, the term “cannula” may be substituted therefore. The sheath 28 may have a hollow tube 30 with a central lumen 32. The tube 30 is coupled to a body 34. In an implementation, the tube 30 is made of stainless steel. In implementations, the tube has a wall thickness of from about 0.2 mm to about 0.5 mm, and more preferably from about 0.3 mm to about 0.4 mm.

The lumen 32 may be sized and dimensioned to closely fit over the shaft 14 of the camera 12. In implementations, the shaft 14 may have a diameter of less than about 20 mm. The diameter of the shaft 14 may be less than about 2.0 mm and may be less than about 1.9 mm. The lumen may have a diameter of less than about 21 mm. The lumen may have a diameter of less than about 2.1 mm. The lumen may have a diameter of less than about 2.0 mm. The diameter of the lumen 32 may be selected based on the outer diameter of the shaft 14.

The tube 30 has a proximal portion 36 coupled to the body 34. The proximal portion may be straight and may be rigid. The tube 30 may also have a bend 38 and a distal portion 40 distal to the bend. The distal portion may be straight and rigid. The distal portion 40 may have a plurality of openings for fluid communication. The plurality of openings may be arranged to direct fluid in front of the tube 30 to clear a path in front of the tube. The tube 30 may have an overall length of from about 76 mm to about 153 mm, and more preferably from about 89 mm to about 115 mm, and more preferably, from about 104 mm to about 107 mm.

The distal portion 40 may have a longitudinal axis at an angle of between about 5 degrees and about 90 degrees relative to a longitudinal axis of the proximal portion 36 of the tube 30. The distal portion 40 may have a longitudinal axis at an angle of about 30 degrees, about 45 degrees or about 70 degrees relative to a longitudinal axis of the proximal portion 36 of the tube 30. The distal portion 40 may have a longitudinal axis at an angle of between about 7.5 degrees and about 15 degrees and more preferably between about 9 degrees and about 11 degrees relative to a longitudinal axis of the proximal portion 36 of the tube 30.

The bend 38 may have a radius of from about 12 mm to about 38 mm, and may have a radius of from about 25 mm to about 32 mm, and may have a radius of from about 25 mm to about 28 mm. The distal portion 40 may have a length of from about 12 mm to about 38 mm, and may have a length of from about 17 mm to about 23 mm. The camera shaft 14 may not be fully rigid and may have flexible or semi-rigid characteristics allowing it to bend/deflect around the bend 38 of the sheath 28. The shaft 14 may be made of stainless steel. In additional implementations, the shaft 14 may be made of a plastic such as polycarbonate, Delrin® by DuPont, Radel® by Solvay, or Ultem® by SABIC.

The body 34 is configured to allow insertion of the camera shaft 14. The body 34 may be configured to removably couple to the handpiece 16. The body 34 may have a port 42 for coupling a fluid source or a suction source. In an implementation, the port 42 has a luer connection that may be capped when not in use. The body 34 may have a plurality of ports 42 with one port being coupled to a fluid source and another port being coupled to a suction source. The body 34 may also have a mechanism for coupling the body to the shaft 14 and the handpiece 16 to set the shaft in a particular position along the longitudinal axis of the tube 30 while allowing for rotation of the sheath 28 relative to the camera 12.

As shown in FIG. 6, the body 34 may have a plurality of detents 70 and the handpiece 16 may have a plurality of projections 72. The projections 72 may be configured to movably fit inside the detents 70 to stabilize the position of the camera 12 relative to the sheath 28 and to prevent accidental rotation. At least one grommet, washer or dam may be placed inside the body 34 to form a seal between the body and the handpiece 16 to prevent liquid leaking from an interface between the body and the handpiece.

In additional implementations, the body 34 may have at least one handle for rotating the sheath 28 relative to the camera 12. In an additional implementation, the outside of the tube 30 may have a coating for lubricating the insertion of the tube into a patient. In an additional implementation the inside of the tube 30 may have a coating for, for example, lubricating movement and rotation of the camera shaft 14 within the tube.

Preferably, once the camera shaft 14 is inserted into the sheath 28, the sheath is rotatable relative to the camera 12 such that the direction of view may be manipulated by moving the sheath without changing the position of the image sensor 26 in the camera. In an implementation the camera shaft 14 may be moved axially relative to the sheath 28 so that the shaft 14 variably protrudes from the distal portion 40 of the sheath for differential visualization of a surgical field.

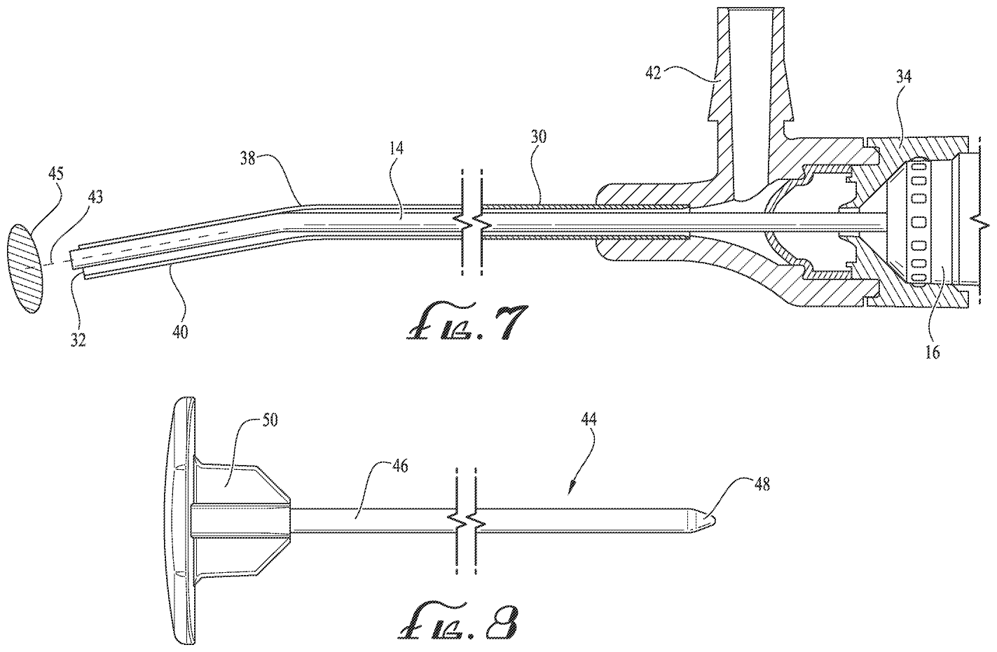

As shown in FIG. 7, when the camera 12 is inserted in the sheath 28, the shaft 14 elastically deforms to the bend 38 and distal portion 40 of the sheath. This deflection of the shaft 14 changes an axis of view 43 and a field of view 45 for the camera. As the sheath 28 is rotated relative to the camera 12, the direction of the deflection changes, which changes the field of view 45. Once inserted into the sheath 28, a portion of the shaft 14 is concentrically positioned within the proximal portion 36 of the sheath.

For example, the sheath 28 may be used with a 0 degree endoscopic camera to create a non-zero degree angle of view. If the distal portion 40 of the sheath 28 has an angle of 10 degrees relative to the proximal portion 36, then the sheath may be used to make a 0 degree endoscopic camera a 10 degree endoscopic camera. Because the camera 12 may be held steady, the image sensor 26 may also be held steady. Rotation of the bent sheath 28 while holding the camera 12 steady moves the direction of view without rotating the sensor 26. This may prevent an operator, such as a surgeon, from becoming disoriented.

The sheath 28 may be coupled to the camera 12. The shaft 14 may have a longitudinal axis 25. The longitudinal axis may correspond to the center of the field of view of the sensor 26. The proximal portion 30 of the shaft 14 may have a longitudinal axis 31. The sheath 28 may be coupled to the camera 12 such that the longitudinal axis 31 and the longitudinal axis 25 overlap or may be the same. The sheath 28 may be configured with the body 34 such that the sheath rotates about the longitudinal axis 31. In some implementations, the sheath 28 may be coupled to the camera 12 such that the sheath rotates about the longitudinal axis 25 of the camera 12. Accordingly, the sheath 28 may rotate about the center of the sensor 26. The shaft 14 may retain a constant rotational angle relative to the handpiece 16. At the same time, the field of view 45 of the sensor 26 may be reoriented by the distal end of the sheath 28.

A plane of rotation may be approximately perpendicular the longitudinal axis of the sheath 28 or shaft 14. In some implementations, the proximal portion 36 of the sheath 28 may be concentric with the shaft 14. As the sheath 28 rotates about the shaft 14, the distal portion 40 of the sheath 28 may interfere with the shaft 14 due to the angle of the distal portion 40 relative to the proximal portion 36. The interference of the sheath 28 with the shaft 14 may cause the shaft 14 to bend or deflect orienting the camera at a viewing angle greater than 0 degrees and which may include the angles and ranges described elsewhere in this application, for example, between 10 and 70 degrees, and particular examples at 30, 45, and 70 degree viewing angles (e.g. from the longitudinal axis of the sheath 28 or shaft 14).

The shaft 14 may be substantially rigid and may be substantially straight prior to insertion into the sheath 28. In an implementation, the shaft 14 returns to a substantially straight and substantially rigid configuration upon removal from the sheath 28. As such, the shaft 14 may elastically deformable. In some implementations, the shaft 14 does not return to a substantially straight and substantially rigid configuration upon removal from the sheath 28.

The shaft 14 may be a unitary piece and formed of a single material. In some implementations, the shaft 14 may be formed of multiple materials. For example, the shaft 14 may include one or more flexible portions and one or more non-flexible portions. The one or more flexible portions may elastically deform when the shaft 14 interferes with the sheath 28.

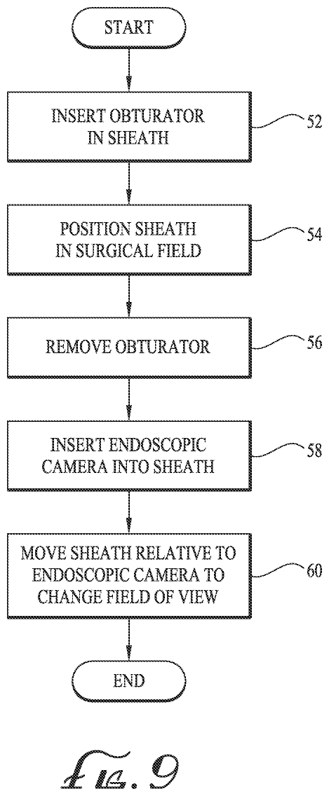

With reference to FIG. 8, an obturator 44 insertable in the sheath 28 will now be described. The obturator 44 has a shaft 46 configured to fit inside the tube 30. In an implementation, the shaft 44 has a point 48 at it distal tip to help insertion of the sheath 28. In other implementations, the shaft 44 has a blunt distal tip. The obturator has a handle 50 coupled a proximal end of the shaft 46. The handle 50 assists with placement of the obturator 44 in the sheath 28 and placement of the sheath in a desired surgical field.

The shaft 46 may be flexible enough to bend or deflect around the bend 38 of the sheath 28. In implementations, the shaft 46 is made of a plastic such as Delrin® by DuPont, Radel® by Solvay, or Ultem® by SABIC. The shaft 46 is configured to fit inside the tube 30. In implementations, the shaft has a diameter of less than about 20 mm, preferably less than about 2.0 mm and more preferably less than about 1.9 mm.

With reference to FIG. 9, the present disclosure also provides a method for conducting endoscopic surgery using an endoscopic camera and a sheath. The obturator 44 is inserted into the sheath 28, step 52. The obturator may extend a short distance from a distal end of the sheath for puncturing a patient's skin and providing a route for insertion of the sheath. The sheath 28 is then advanced to a desired surgical field, step 54. Once the sheath 28 is in position, the obturator 44 is removed, step 56. After the obturator 44 has been removed, camera 12 is inserted into the sheath 28, step 58. The sheath 28 may be manipulated relative to the camera 12 to change the field of view, step 60. Once the endoscopic camera is no longer needed in the surgical field, the camera 12 and sheath 28 may be removed.

In some implementations, the camera 12 rather than the obturator 44 is inserted into the sheath 28 as the sheath is introduced into a surgical field. Additionally, a fluid source may be coupled to the port 42 for providing fluid to the surgical field. Additionally, a suction source may be coupled to the port 42 for removing fluid or other material from the surgical field.

There is disclosed in the above description and the drawings, a surgical imaging system and method that fully and effectively overcomes the disadvantages associated with the prior art. However, it will be apparent that variations and modifications of the disclosed implementations may be made without departing from the principles of the invention. The presentation of the implementations herein is offered by way of example only and not limitation, with a true scope and spirit of the invention being indicated by the following claims.

Any element in a claim that does not explicitly state “means” for performing a specified function or “step” for performing a specified function, should not be interpreted as a “means” or “step” clause as specified in 35 U.S.C. § 112.

What is claimed is:

1. An endoscopic camera system comprising:
a camera further comprising a shaft coupled to a handpiece; and
a sheath further comprising a hollow tube coupled to a body, the tube further comprising:
a proximal portion coupled to the body;
a bend at a distal end of the proximal portion; and
a distal portion distal to the bend; and
wherein the sheath is configured to deflect the shaft and wherein the sheath is rotatable about the shaft to change a direction of the deflection.

2. The endoscopic camera system of claim 1 wherein the shaft further comprises a proximal end and a distal end and wherein an image sensor is positioned proximal to the distal end.

3. The endoscopic camera system of claim 2 wherein the shaft further comprises optics to provide light to the image sensor and wherein the optics provide a field of view approximately along a longitudinal axis of the shaft.

4. The endoscopic camera system of claim 1 wherein:
the shaft has a diameter of less than about 2.0 mm and the hollow tube further comprises a lumen with a diameter of less than about 2.1 mm.

5. The endoscopic camera system of claim 1 wherein the distal portion has a longitudinal axis at an angle of between about 5 degrees and about 90 degrees relative to a longitudinal axis of the proximal portion.

6. The endoscopic camera system of claim 1 wherein the distal portion has a longitudinal axis at an angle of between about 7.5 degrees and about 15 degrees relative to a longitudinal axis of the proximal portion.

7. The endoscopic camera system of claim 1 wherein the distal portion has a longitudinal axis at an angle of between about 9 degrees and about 11 degrees relative to a longitudinal axis of the proximal portion.

8. The endoscopic camera system of claim 1 wherein the bend has a radius of from about 12 mm to about 38 mm.

9. The endoscopic camera system of claim 1 wherein the bend has a radius of from about 25 mm to about 32 mm.

10. The endoscopic camera system of claim 1 further comprising a flexible obturator configured to be removably inserted into the sheath.

11. A sheath for an endoscopic camera system comprising a body and a hollow tube, the hollow tube further comprising:
a proximal portion coupled to the body;
a bend at a distal end of the proximal portion; and
a distal portion distal to the bend.

12. The sheath of claim 11 wherein the hollow tube comprises a lumen with a diameter of less than about 2.1 mm.

13. The sheath of claim 11 wherein the distal portion has a longitudinal axis at an angle of between about 5 degrees and about 25 degrees relative to a longitudinal axis of the proximal portion.

14. The sheath of claim 11 wherein the distal portion has a longitudinal axis at an angle of between about 7.5 degrees and about 15 degrees relative to a longitudinal axis of the proximal portion.

15. The sheath of claim 11 wherein the distal portion has a longitudinal axis at an angle of between about 9 degrees and about 11 degrees relative to a longitudinal axis of the proximal portion.

16. The sheath of claim 11 wherein the bend has a radius of from about 12 mm to about 38 mm.

17. The sheath of claim 11 wherein the bend has a radius of from about 25 mm to about 32 mm.

18. A method for imaging a surgical field with a camera further comprising a shaft coupled to a handpiece and a sheath further comprising a hollow tube coupled to a body, the tube further comprising a proximal portion coupled to the body; a bend at a distal end of the proximal portion;
and a distal portion distal to the bend, the method further comprising the steps of:
positioning the sheath in a surgical field;
inserting the camera shaft into the sheath to deflect the shaft; and
moving the sheath relative to the shaft to change an angle of the deflection to change the field of view.

19. The method of claim 18 further comprising the steps of:
inserting an obturator into the sheath prior to positioning the sheath in a surgical field; and
removing the obturator from the sheath prior to inserting the camera shaft into the sheath.

20. The method of claim 18 further comprising connecting at least one of a fluid source and a suction source to the sheath.
